(19) United States
(12) Patent Application Publication
(10) Pub. No.: US 2007/0122387 A1
(43) Pub. Date: May 31, 2007

(54) Hair grafts derived from plucked hair

(75) Inventors: Stephen A. Cochran, Tucker, GA (US); Bryan Marshall, Atlanta, GA (US); Thomas H. Barrows, Austell, GA (US); Yandong Su, Atlanta, GA (US); Robyn Schlicher, Atlanta, GA (US)

Correspondence Address: MICHAEL BEST & FRIEDRICH, LLP, ONE SOUTH PINCKNEY STREET, P O BOX 1806, MADISON, WI, 53701, US

(73) Assignee: Aderans Research Institute, Inc., Beverly Hills, CA (US)

(21) Appl. No.: 11/603,818

(22) Filed: Nov. 22, 2006

Related U.S. Application Data
(60) Provisional application No. 60/738,881, filed on Nov. 22, 2005.

(51) Int. Cl. A61K 35/36

(52) U.S. Cl. 424/93.7

(57) ABSTRACT

The present invention is a hair graft derived from a plucked hair comprising a plucked hair having adhered epidermal stem cells and associated follicular dermal cells. The present invention also includes methods of making a hair graft, methods of implanting a hair graft and methods of identifying inductive follicular dermal cells.

### CROSS-REFERENCE TO RELATED APPLICATIONS

This application claims benefit of priority under 35 U.S.C. § 119 from U.S. Provisional Application Ser. No. 60/738,881, filed Nov. 22, 2005. The application is incorporated herein by reference in its entirety.

### BACKGROUND

Hair loss may occur due to a variety of conditions and may affect anyone: men, women and children. Hair loss conditions include, but are not limited to, alopecia capitis totalis, i.e., loss of all scalp hair, alopecia universalis, i.e., loss of hair over the whole body, alopecia areata, i.e., patchy hair loss, and androgenetic alopecia, i.e., male pattern baldness. Medications are available to treat alopecia including minoxidil, finasteride, corticosteroids and anthralin. However, discontinuing use of the medication often stops any new hair growth resulting from the medication because the medication does not cause new follicles to be produced.

More aggressive hair restoration methods include hair transplants and scalp reduction surgery. Hair transplantation entails excision of a full-thickness strip of scalp tissue from the back of the head and carefully dissecting it into hundreds of “follicular unit grafts,” each containing one to several hairs and implanting the grafts into the bald sections of the scalp where an equal number of recipient sites have been created by making stab wounds with a pointed blade. Hair transplantation creates no new hair follicles and often not all of the explanted follicles successfully transplant. Scalp reduction surgery, which is becoming less popular, aims to surgically reduce the area of bald skin on subject's head. Both hair transplantation and scalp reduction surgery are expensive and may be painful. Moreover, both carry possible risks of infection and scarring.

It is well known that specific cells within the hair follicle, including epidermal stem cells and dermal papilla cells, have the capacity to induce follicle neogenesis i.e., new follicle formation. Attempts have been made to exploit the inductive capabilities of these cells, including injecting follicular dermal cells directly into the skin and implanting plucked hairs carrying epithelial cells having various proliferative and differentiative characteristics. These previous attempts at producing follicle neogenesis have failed to produce reliable, reproducible and cosmetically satisfactory results.

### SUMMARY

The present invention relates to a hair graft derived from at least one plucked hair and methods of implanting the hair graft. The graft of the present invention suitably comprises at least one plucked hair from a subject. The at least one plucked hair has adhered epidermal stem cells derived from the hair follicle from which the hair was plucked, and follicular dermal cells are associated with the adhered epidermal stem cells.

Suitably, the hair graft may be contained within a bioabsorbable scaffold. Further, the bioabsorbable scaffold may have moieties associated with the scaffold that facilitate or enhance the growth of new hair. The bioabsorbable scaffold of the hair graft may be a hollow filament. The hollow filament may contain a protectant contained within the inner lumen of the hollow filament.

In another embodiment the invention provides a method of making a hair graft. The hair graft suitably comprises at least one plucked hair having adhered epidermal stem cells and follicular dermal cells associated with the epidermal stem cells. The hair graft may be contained within a bioabsorbable scaffold.

In a further embodiment, the invention provides a method of implanting a hair graft by creating a wound in skin of a subject and implanting a plucked hair graft having adhered epidermal stem cells and follicular dermal cells associated with the epidermal stem cells. The hair graft may be implanted while contained in a bioabsorbable scaffold. Further, the hair graft suitably may be implanted in a wound containing a protectant.

In an additional embodiment, the present invention provides a method of implanting a hair graft using a hypodermic needle and syringe. Suitably, the syringe may be loaded with a protectant. The hair graft and protectant may suitably be injected into a subject.

In another embodiment, the present invention provides a method for identifying inductive follicular dermal cells comprising incubating at least one plucked hair having adhered epidermal stem cells with follicular dermal cells. The method may be used to test a plurality of cells and select a sub-population of cells capable of inducing follicular neogenesis. The cells of the sub-population may be capable of inducing follicular neogenesis alone or in conjunction with other cell types. The self-selected sub-population may be multiplied.

Other aspects of the invention will become apparent by consideration of the detailed description and accompanying drawings.

### BRIEF DESCRIPTION OF THE DRAWINGS

FIG. 1 is a confocal photomicrograph of a plucked hair having adhered epidermal stem cells. The epidermal stem cells are revealed by their uptake of a fluorescently labeled antibody to the CD200 cell surface protein.

FIG. 2 is a confocal photomicrograph of a plucked hair that has been incubated in vitro with follicular dermal cells double labeled with fluorescently labeled antibodies to the CD73 (orange) and CD90 (green) cell surface proteins. CD90 labels all types of dermal cells. CD73 is a known marker for mesenchymal stem cells.

FIG. 2a (inset) is a confocal photomicrograph showing the population of cells in which the plucked hair of FIG. 2 was incubated.

FIG. 3 is a photograph of the underside of skin of a SCID mouse at the site of implantation of a traditional human scalp follicular unit hair graft comprised of 2 hairs.

FIG. 3a (inset at right) is a photograph of the same two hair shafts growing from the human hair graft implantation as seen on the surface of the SCID mouse skin.

FIG. 3b (inset at left) is a photograph of a typical plucked hair having adhered epidermal stem cells.

FIG. 4 is a photograph of a plucked hair prior to implantation partially loaded into a 27 gauge hypodermic needle. The protruding end of the hair in the needle has a “club” end. Several other hairs with similar club ends are shown after implantation where the club ends are now buried beneath the skin.

FIG. 5 is a photograph of the hypodermic needle, loaded with a plucked hair and saline solution that has been injected into the skin and withdrawn slightly.

FIG. 6 is a photograph of a plucked hair that has been properly implanted.

FIG. 7 is an H&E stained histological section of biopsied pig skin at the site where trichogenic newborn pig (same-breed) skin cells were implanted at the interface between the fat and the dermis. A plucked hair devoid of living cells was implanted with the trichogenic cells.

FIG. 8 is a photograph of an implanted plucked hair, having adhered epidermal stem cells and associated follicular dermal cells, placed under the skin of a nu/nu mouse for 20 weeks.

### DETAILED DESCRIPTION

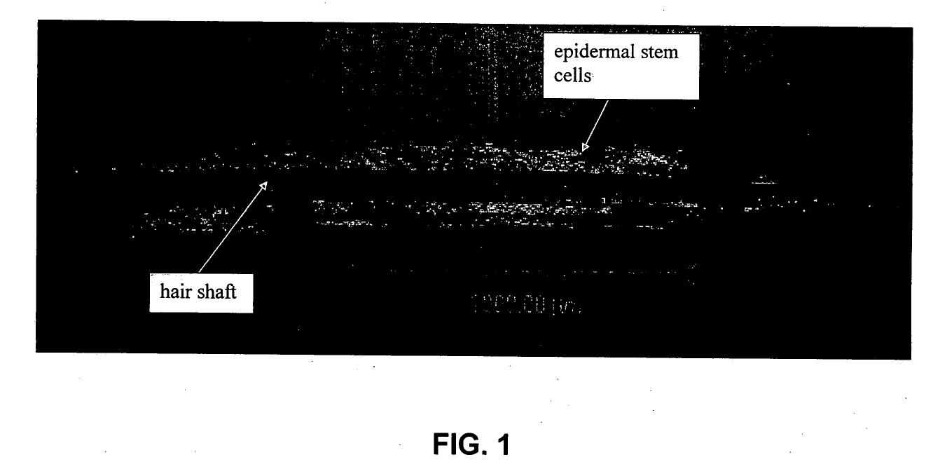

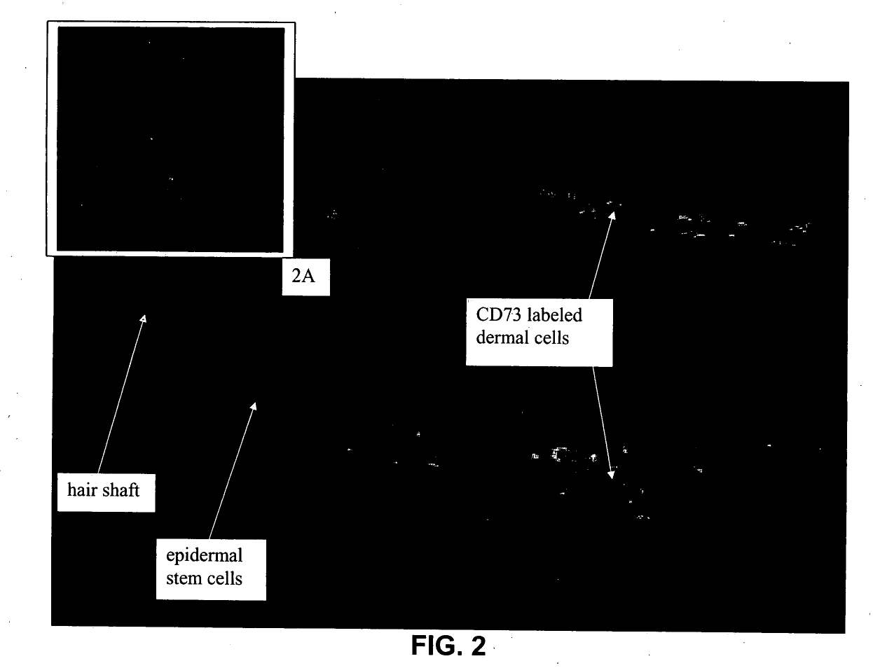

The present invention relates to a hair graft and methods of making and implanting a hair graft. In one embodiment, the hair graft comprises at least one plucked hair having adhered epidermal stem cells and follicular dermal cells not natively associated with the epidermal stem cells. Follicular dermal cells include, but are not limited to, dermal stem cells, dermal papilla and dermal sheath cells, dermal fibroblasts, mesenchymal stem cells, and other cells found in the hair follicle capable of inducing follicle neogenesis. The adhered epidermal stem cells are from the hair follicle from which the hair was plucked. See FIG. 1. Suitably, the plucked hair having adhered epidermal stem cells is incubated with a population of follicular dermal cells. During incubation, the follicular dermal cells suitably become associated with the epidermal stem cells. See FIG. 2. The term “associated with” refers to the physical relationship between the epidermal stem cells and the follicular dermal cells. The term “associated with”includes, but is not limited to, a physical relationship between the cell types that demonstrates attachment, contact, connection, closeness, adherence, binding, or affiliation.

In a further embodiment, the hair graft further comprises a bioabsorbable scaffold. Suitably, the bioabsorbable scaffold is a hollow filament having an inner lumen. The hollow filament has a first end and a second end. The hollow filament may have one closed end, either the first end or the second end. Alternatively, the hollow filament has two open ends, both the first end and the second end. In a suitable embodiment, the at least one plucked hair is located within the inner lumen of the hollow filament.

The term “bioabsorbable” refers to any material the human body can break down into non-toxic by-products that are excreted from the body or metabolized therein. Suitable bioabsorbable materials for manufacture of the scaffold include, but are not limited to, poly(lactic acid), poly(glycolic acid), poly(trimethylene carbonate), poly(dimethyltrimethylene carbonate), poly(amino acids)s, tyrosine-derived poly(carbonates)s, poly(carbonates)s, poly(caprolactone), poly(para-dioxanone), poly(esters)s, poly(ester-amides)s, poly(anhydrides)s, poly(ortho esters)s, collagen, gelatin, serum albumin, proteins, polysaccharides, mucopolysaccharides, carbohydrates, glycosaminoglycans, poly(ethylene glycols)s, poly(propylene glycols)s, poly(acrylate esters)s, poly(methacrylate esters)s, poly(vinyl alcohol), hyaluronic acid, chondroitin sulfate, heparin, dermatan sulfate, versican, copolymers, blends and mixtures of polymers, and oligomers containing bioabsorbable linkages.

For example, hyaluronic acid may be converted into an insoluble crosslinked material (“HAX”) by treatment with a condensing agent, suitably 1-ethyl-3-(3-dimethylaminopropyl) carbodiimide (“EDC”). Alternatively, hyaluronic acid may be converted into an insoluble material by esterification, e.g., forming the benzyl ester of hyaluronic acid, and used to prepare the bioabsorbable scaffold. Suitably, trans-esterification crosslinked HAX is an insoluble material used because the resultant product is then converted back into a soluble hyaluronic acid upon hydrolysis of the ester linkages. Hydrolysis of the ester linkages usually takes place within a few days in vivo. Various crosslinking agents may be employed in the preparation of the bioabsorbable scaffold including, but not limited to, aliphatic diamines, diaminoacid esters such as alkyl esters of lysine, and amine-terminated poly(ethylene glycol).

Various molecular moieties may be associated with the bioabsorbable scaffold using, for example, surface modifications, graft polymerization, copolymerization of bioabsorbable materials or blending of at least one moiety and the bioabsorbable material(s) used in forming the bioabsorbable scaffold. Suitable moieties include, but are not limited to, growth factors, angiogenesis factors, cell attachment binding site moieties, cell signaling molecules, other small molecules, e.g., drugs that enhance hair follicle regrowth such as monoxidil, glycoproteins, e.g., chondroitin sulfate, dermatan sulfate, and versican, other bioactive molecules or combinations thereof.

The term “growth factor” refers to a naturally occurring protein capable of stimulating cellular proliferation and cellular differentiation. Growth factors are important for regulating a variety of cellular processes. Well-known growth factors suitable for use in the present invention include, but are not limited to, granulocyte-colony stimulating factor (“G-CSF”), granulocyte-macrophage colony stimulating factor (“GM-CSF”), nerve growth factor (“NGF”), neutrophins, platelet-derived growth factor (“PDGF”), erythropoietin (“EPO”), thrombopoietin (“TPO”), myostatin (“GDF-8”), growth differentiation factor-9 (“GDF9”), basic fibroblast growth factor (“bFGF” or “FGF2”), epidermal growth factor (“EGF”), placenta derived growth factor (“PLGDF”), and hepatocyte growth factor (“HGF”).

Similarly, the term “angiogenesis factor” refers to a naturally occurring protein capable of stimulating angiogenesis. Suitable angiogenesis factors for the present invention include, but are not limited to, vascular endothelial growth factor (“VEGF”), endothelial cell stimulating angiogenesis factor (“ESAF”) and any nonmitogenic angiogenesis factors present in wound fluid.

The term “cell attachment binding site moiety” refers to a protein that plays a role in cell-cell/cell-matrix interaction and cellular communications. Examples of suitable cell attachment binding site moieties include, but are not limited to, integrins, cadherins, cell adhesion molecules (“CAMs”) and selectins.

The term “cell signaling molecule” refers to a chemical involved in transmitting information between cells. Such molecules are released from the cell sending the signal by crossing over the gap between cells, interacting with receptors in another cell, and triggering a response in that cell. Cell signaling molecules naturally are part of a complex system of communication that governs basic cellular activities and coordinates cell actions.

The term “bioactive molecule” refers to any molecule that has pharmacological activity that is beneficial to hair follicle neogenesis and survival. Suitable bioactive molecules may include, but are not limited to, cell signaling agonists or antagonists.

Association of at least one moiety with the bioabsorbable scaffold may suitably be advantageous for improved association between the epidermal stem cells and the follicular dermal cells and/or improved cell function, cell aggregation or cell initiation of the follicle neogenesis process. Associated moieties, such as growth factors and angiogenesis factors, may be released during the degradation of the bioabsorbable scaffold and encourage blood vessel growth into the newly forming follicle. Higher molecular weight moieties, such as proteins, glycoproteins, and other biopolymers, such as collagen, laminin, and fibronectin, may be covalently or electrostatically associated with the bioabsorbable scaffold to suitably provide greater physical integrity, cell attachment capacity, or bioactivity. For example, covalent attachment of bioactive molecules to a hyaluronic acid structure suitably enhances the performance of the resultant scaffold. Peptides containing the cell attachment domain amino acid sequence Arg-Gly-Asp (RGD) may be used to aid follicular dermal papilla cell attachment to the scaffold.

In another embodiment, the bioabsorbable scaffold may contain a protectant. The protectant may completely fill the bioabsorbable scaffold. The term “protectant” refers to any substance that serves to protect cells from trauma associated with implantation or destruction by the inflammatory process of wound healing. Alternatively, the protectant may only partially fill the inner lumen of the bioabsorbable scaffold, e.g., about 10% full, about 25% full, about 50% full. In another alternative, the protectant may coat all or a portion of the inner wall of the filament. The protectant may fill the bioabsorbable scaffold from between about 0% to about 100%.

Many commercially and clinically available substances may be used as a protectant. Suitably, the protectant is bioabsorbable and may be, but is not limited to, a fibrous material, a gel-forming material or a porous material. The protectant includes, but is not limited to, collagen, gelatin, cellulose, starch, dextrin, chitosan, lipoproteins, recombinant human forms of collagen and gelatin, fibrinogen, fibrin, fibronectin, laminin, albumin, serum proteins, polysaccharides, mucopolysaccarides, chondroitin sulfate solution, hyaluronic acid, biopolymers that naturally occur in the body or combinations thereof. The protectant may be used either in native form or in a modified form, e.g., crosslinked with pharmaceutically acceptable crosslinking agents, which alters the characteristics of the protectant, e.g., to reduce solubility. The protectant may also be combined with a suspension of follicular dermal cells.

A most suitable protectant is autologous serum and/or plasma from the subject in which the hair graft is implanted. Autologous serum and/or plasma may be obtained by drawing a small amount of whole blood from the subject and removing the cells by centrifugation. Advantages of using autologous serum and/or plasma include providing an anchor for the hair graft via natural clotting properties associated with the serum and/or plasma. Also, the autologous serum and/or plasma may contain nutrient molecules and other native beneficial factors to further nurture follicle neogenesis.

In a further embodiment, the protectant is a gel-forming material. For example, the gel-forming material may be a copolymer of ethylene oxide and propylene oxide, e.g., PLURONIC™ F-127 (BASF Corporation, Mount Olive, N.J.), that is compatible with living cells and above a critical concentration forms a gel when warmed to, e.g., body temperature, from cooler temperatures. Furthering the example, the bioabsorbable scaffold may be first treated with the copolymer in alcohol followed by evaporation of the alcohol to impart a suitable hydrophilic coating on the inner lumen. A cold solution of the copolymer containing a suspension of follicular dermal cells may then be wicked or injected into the inner lumen. The bioabsorbable scaffold, now containing a mixture of the protectant and the follicular dermal cells, may then be warmed to induce gelling of the copolymer. Suitably, the gel formation prevents the follicular dermal cells from being dislodged from the bioabsorbable scaffold. Further, the at least one plucked hair having adhered epidermal stem cells may then be introduced to the follicular dermal cells located within the bioabsorbable scaffold to allow for the association of the epidermal stem cells and the follicular dermal cells. Alternative suitable gel-forming materials include, but are not limited to, collagen, gelatin, albumin, laminin, heparin sulfate proteogylcans, entactin, ethylene oxide, propylene oxide, MATRIGEL™ basement membrane matrix (BD Biosciences, San Jose, Calif.), polyethylene glycol molecules with end groups that covalently react to form gel networks or combinations thereof.

In another embodiment, the present invention includes a method of making a hair graft. Suitably, at least one hair is plucked from a subject, the at least one plucked hair having adhered epidermal stem cells. The plucking of hairs does not result in permanent hair loss because the portion of the follicle that remains in the skin after plucking is able to grow new hair, in contrast to traditional hair transplantation in which donor follicles are permanently removed from the donor site. Suitably, the at least one plucked hair is then incubated with cultured follicular dermal cells. The follicular dermal cells may come from the same subject as the plucked hair, a different human subject or may be bought. (Cell Applications, Inc., San Diego, Calif.). If the follicular dermal cells are obtained from a human subject, a sacrifice of some donor follicles is necessary. However, the follicular dermal cells may suitably be cultured such that only a few follicles may produce multiple hair grafts.

During incubation, the follicular dermal cells become associated with the epidermal stem cells adhered to the plucked hair. Surprisingly, it has been discovered that a sub-population of follicular dermal cells having the most potent follicle-inductive properties are selectively attracted to the epidermal stem cells present on the outermost surface of the plucked hair. See FIG. 2. Optionally, the at least one plucked hair and the follicular dermal cells may be incubated in a scaffold. Suitably, the scaffold is bioabsorbable. However, non-bioabsorbable scaffolds may be used prior to implantation into a subject. Further, the scaffold may contain a protectant as described above. Additionally, the follicular dermal cells may be located within the protectant to facilitate association of the follicular dermal cells with the epidermal stem cells adhered to the plucked hair.

In a further embodiment, the present invention provides a method of implanting a hair graft comprising at least one plucked hair having adhered epidermal stem cells and associated follicular dermal cells. Suitably, a wound is created in the skin of a subject and the hair graft is then implanted. Alternatively, a protectant, as described above, may be placed in the wound prior to, during or following implantation of the hair graft. Additionally, the hair graft of the method may be incubated in a bioabsorbable scaffold. Suitably, if the hair graft is incubated in a bioabsorbable scaffold it may then be implanted using the traditional wound method, described above, as is currently done with traditional grafts. Alternatively, the hair graft and bioabsorbable scaffold combination may be implanted using the “stick and place” method of grafting. In the “stick and place” method, the skin is pierced with the sharp point of a hollow needle or tube that also serves as a container for the bioabsorbable scaffold ensheathed hair graft. The tube is then inserted into the wound and withdrawn against a push rod that prevents the graft from coming out of the tube and ensures correct placement of the graft. A modification of the “stick and place” method may use a tool such as the Choi implanter, which requires breaking the skin with a pointed instrument prior to inserting the tube and depositing the implant.

Bioabsorbable scaffolds used in methods of the invention may be manufactured from a variety of materials as discussed above. Further, the bioabsorbable scaffold may have at least one moiety associated with it. The associated moiety or moieties may include, but are not limited to, growth factors, angiogenesis factors, cell attachment binding site moieties, cell signaling molecules, small molecules, polypeptides, glycoproteins, bioactive molecules or combinations thereof.

Alternatively, a hair graft may be implanted using a hypodermic needle and a syringe. Suitably, the hypodermic needle used may range from a 18-gauge to a 30-gauge needle depending on the coarseness of the plucked hair and the number of plucked hair grafts that are desired to be simultaneously implanted by loading into the same needle. More suitably, the hypodermic needle used may range from a 25-gauge to a 29-gauge needle. Most suitably, the hypodermic needle used is a 27-gauge needle. A syringe may suitably be loaded with a protectant. The hair graft comprising a plucked hair having adhered epidermal stem cells and associated follicular dermal cells is suitably loaded into the hollow, pointed end of the needle distal to the syringe. Alternatively, the plucked hair may be loaded into the proximal end of the needle, nearest the syringe, prior to attachment of the syringe. Suitably, if the plucked hair is loaded into the proximal end, the end of the hair graft having adhered cells would be loaded first in preparation for implantation. The loading of the protectant into the syringe may take place prior to, concomitant with, or following the loading of the hair into the hypodermic needle. Suitably, the syringe and needle are separated during the respective loading procedures. Alternatively, the syringe may be attached to the hypodermic needle prior to loading, during loading or following loading of the protectant. Similarly, the hypodermic needle may be attached to the syringe prior to loading, during loading or following loading of the hair graft. Regardless of the orientation during the loading process, prior to implantation the needle and syringe suitably are connected. Alternatively, a permanently attached hypodermic needle and syringe may be used.

Following loading of both the hypodermic needle and the syringe, the hypodermic needle may then be inserted into skin of a subject. Most suitably, the needle is inserted through the epidermal and dermal layers of skin and just into the uppermost fat layer. The needle may then be withdrawn from the skin while applying pressure on the syringe to inject the hair graft and protectant. Once the needle is fully removed and excess protectant is expressed, the graft is securely implanted in the skin. Suitably, the epidermal cells and associated follicular dermal cells of the hair graft are at the level of the dermis-fat interface. Hair graft implantation using a syringe and hypodermic needle serves to deliver the graft gently and with minimal trauma. Moreover, using a hypodermic needle/syringe for implantation affords greater control over the direction and angle of the hair shaft egress from the skin, which in turn dictates the orientation of the new follicle that is subsequently formed.

In another embodiment, the present invention provides a method for identifying inductive follicular dermal cells comprising incubating at least one plucked hair having adhered epidermal stem cells in follicular dermal cells. Surprisingly, it has been discovered that a sub-population of follicular dermal cells having the most potent follicle-inductive properties are selectively attracted to the epidermal stem cells present on the outermost surface of the at least one plucked hair. The method may be used to test a plurality of cells and to select a sub-population of cells capable of inducing follicular neogenesis. The cells of the sub-population may be capable of inducing follicular neogenesis alone or in conjunction with other cell types. The self-selected sub-population may be multiplied for use in the previous described hair grafts and methods.

It is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical range recited herein includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification.

The present invention is further explained by the following examples, which should not be construed by way of limiting the scope of the present invention.

### EXAMPLES

### Example 1

Preparation of Collagen/Chondroitin Sulfate Tubes

A 0.48 micrometer diameter stainless steel rod was cut into 2 cm lengths and each piece was dipped into a solution (approximately 10% w/v) of poly(lactic-co-glycolic acid) (“PLGA”) (PURASORB™ PDLG, inherent viscosity 1.06 dl/g in chloroform) dissolved in dimethyl sulfoxide (“DMSO”) (Aldrich Chemical Co., Milwaukee, Wis.). The coated wires were immediately submerged in water whereupon the PLGA came out of solution and deposited on the wires while the DMSO was abstracted by the water and diluted. Hemi-cylindrical cavities (approximately 2 mm wide by 5 mm long and 2 mm deep) and grooves in communication with one end of each cavity to the depth of 1 mm were cut into a block TEFLON™. Collagen (bovine type I, MP Biomedicals, Inc., Aurora, Ohio) was dissolved in 0.05 M acetic acid (0.7% w/v) and placed at each cavity. A PLGA coated wire was also placed in each cavity. A solution of chondroitin-6-sulfate (Sigma Chemical Co., St. Louis, Mo.) dissolved in water (5% w/v) was then placed upon the mold to cover the exposed collagen. After about 20 minutes, the excess chondroitin-6-sulfate solution was decanted and the chondroitin-6-sulfate/collagen coagulum was removed from the mold by pulling up on the wire. The wire was then placed in excess chondroitin-6-sulfate solution to complete the coagulation process. After 20 minutes, the wires with attached coagulum were removed from solution and, holding the uncoated end of the wire with forceps, inserted into a slab of cured silicone rubber so that the coagulum was exposed to air and allowed to dry overnight. This process was then repeated to obtain a second coat of collagen/chondroitin-6-sulfate on the wires. Following the second coat, the wires were then placed in a vial of acetone and allowed to soak overnight to dissolve the PLGA. The collagen/chondroitin-6-sulfate tubes were removed from the wires by grasping the wire firmly at the uncoated end and gently running another forceps along the length of the wire toward the coated end. The resultant tubes were stored under cool, dry conditions until used as scaffolds for hair grafts.

### Example 2

Implantation of Hair Grafts into the Nude (nu/nu) Mouse

A small specimen of whole human scalp tissue from a traditional follicle transplantation donor site was obtained with informed consent from a male patient undergoing hair transplant surgery. The bulbs of the hair follicles contained in this specimen were micro-dissected to obtain the follicular dermal cells, which were then transferred by needle point to culture plates containing small amounts of sterile cell culture media. The follicular dermal cells were allowed to grow and multiply using previously described techniques (see A. G. Messenger, “The culture of dermal papilla cells from human hair follicles,” Br. J. Dermatol. 1984 Jun; 110(6):685-9, the teachings of which are incorporated herein by reference) until a sufficient number of cells became available for experimentation. The cells were then harvested and separated from the supernatant by centrifugation and resuspended in a phosphate buffered saline solution to obtain approximately 100,000 cells per microliter. One microliter of suspended cells were transferred to each of four sections of 0.71 mm inner lumen diameter TEFLON™ tubing that were 6 mm in length.

A different male donor supplied the plucked hairs. The scalp and hair on the back of the head were first disinfected with 70% isopropyl alcohol. Individual hairs were plucked by grasping the base of the shaft with tweezers and quickly pulling. One plucked hair with attached epidermal stem cells was placed in each of the four tubes containing the cultured follicular dermal cells and placed horizontally in an incubator at 37° C. for about 2 hours until follicular dermal cells were seen to be associated with the epidermal stem cells attached to the plucked hair.

An athymic nude mouse was anesthetized and operated upon according to an approved protocol. Small incisions were made using a 19 gauge hypodermic needle in the skin on the back of the mouse and the plucked hairs having adhered epidermal stem cells and associated follicular dermal cells were carefully removed from the TEFLON™ tubes and implanted under the skin, on per incision. At four weeks after implantation, the mice were necropsied and evaluated for follicular neogenesis. Histological analysis of the implant sites revealed the formation of follicular-like structures in each case.

### Example 3

Preparation and Association of Follicular Dermal Cells with Epidermal Stem Cells Adhered to a Plucked Hair

Hairs were plucked using curved hemostats from a region of a donor's scalp that had been disinfected with 70% ethanol. After plucking, the plucked hairs were submerged in a Petri dish containing Dulbecco's Modified Eagles Medium (“DMEM”)/F-12 media with 2% to 3% antibiotic and incubated at 37° C. for at least 30 minutes and up to 1.5 hours. During the incubation, a six-well plate was prepared to receive the plucked hairs by coating the bottom surface of the wells with 5% poly-HEMA in methanol to discourage follicular dermal cell attachment to the growth surface; thus forcing the follicular dermal cells to associate with the epidermal stem cells adhered to the plucked hairs. The poly-HEMA was applied and air dried in a bio-safety cabinet (“BSC”) for at least 30 minutes. Alternatively, the six-well plates were prepared the day prior to the experiment. Human dermal fibroblasts and human dermal papilla cells were purchased from Cell Applications, Inc., San Diego, Calif. In addition, follicular dermal cells were obtained, with informed consent, from follicular papillae dissected from human scalp tissue donated from subjects undergoing hair restorative surgery. Cultured follicular dermal cells were harvested, usually 24 hours prior to use, and stored at 4° C. in DMEM/F-12 media.

The plucked hairs were sorted for suitability based on the size and thickness of the adhered tissue, including the epidermal stem cells. Hairs with no tissue, minimum tissue or those that appeared to have only an inner root sheath (“IRS”) cells were discarded. The cuticles of suitable hairs were trimmed to approximately 3 mm in length. These prepared plucked hairs were then placed in the poly-HEMA treated six-well plates containing 1 ml of DMEM/F-12 media in each well. One ml suspensions of prepared dermal fibroblasts and dermal papilla cells were then added to the wells, raising the final volume to 2 ml. A range of dermal cell concentrations, from 0.5 million to 10 million cells per milliliter, were tested. All steps were performed under aseptic conditions, in a sterile BSC. The six-well plate was covered with a lid and placed in a 37° C. incubator for 30 minutes. Following incubation, the plate was gently agitated using a motorized tilt-rocker plate at room temperature for approximately 4 hours. The resultant plucked hairs, having adhered epidermal stem cells with associated dermal cells, were then implanted in nu/nu mice. In some cases, samples were incubated overnight at either 37° C. or at 4° C. prior to implantation in nude mice.

### Example 4

Fluorescent Labeling

For samples to be studied using confocal microscopy (see FIGS. 1 and 2), antibody labeling of surface receptors was performed. As an initial step, a plucked hair having adhered tissue was fluorescently labeled with CD200. CD200 labels the epidermal stem cells. See FIG. 1. In a separate labeling experiment, a plucked hair having adhered (unlabeled) epidermal stem cells was incubated in a population of follicular dermal cells. See FIG. 2A. The population of follicular dermal cells was double labeled with antibodies CD73 and CD90. CD90 (green) labels all types of dermal cells. CD73 (orange) is a known antibody to mesenchymal stem cells. All antibodies were added at a concentration of 1:200. As can be seen through comparison of FIG. 2 to FIG. 1, the CD73 labeled cells selectively associated with the region on the plucked hair where the epidermal stem cells localize.

### Example 5

Long Term Implantation of Human Plucked Hair Grafts and Follicular Dermal Cell-Seeded Plucked Human Hairs in Nude (nu/nu) and SCID Mice

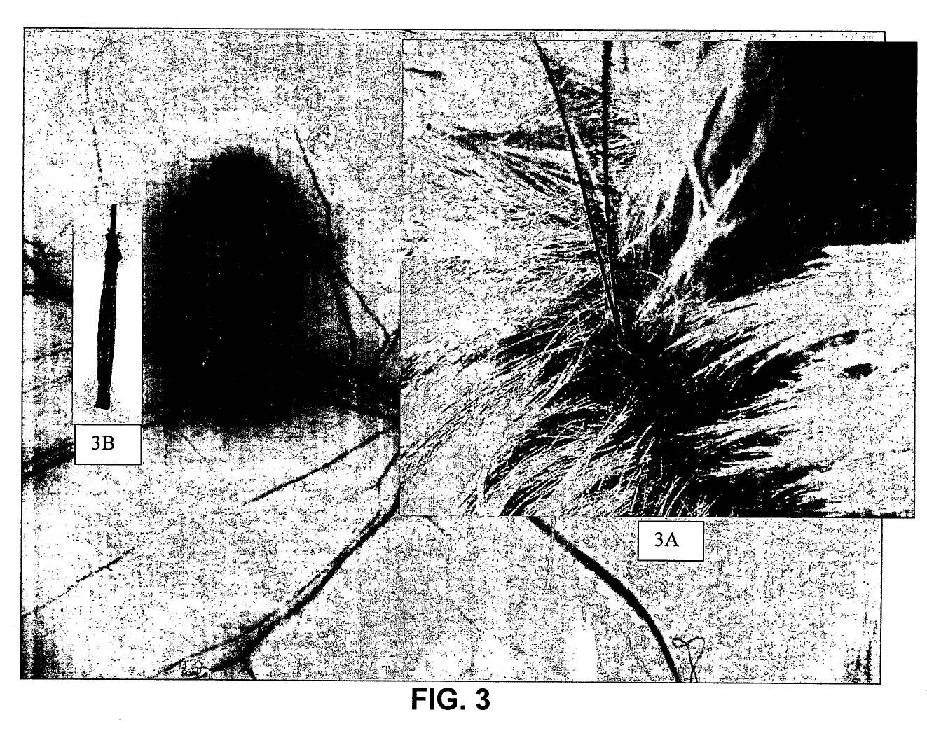

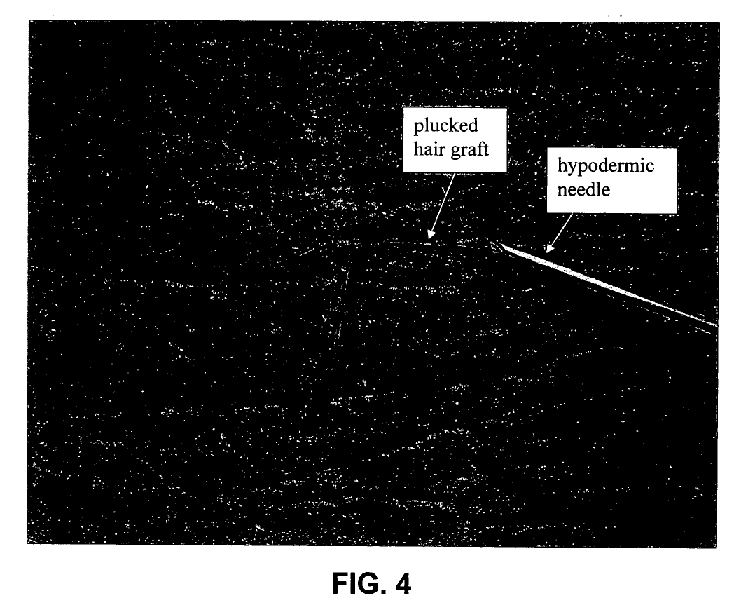

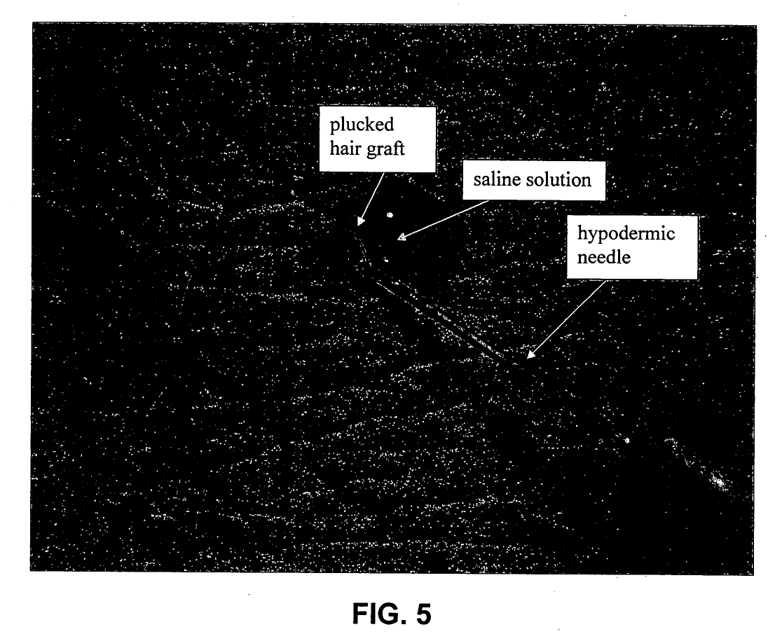

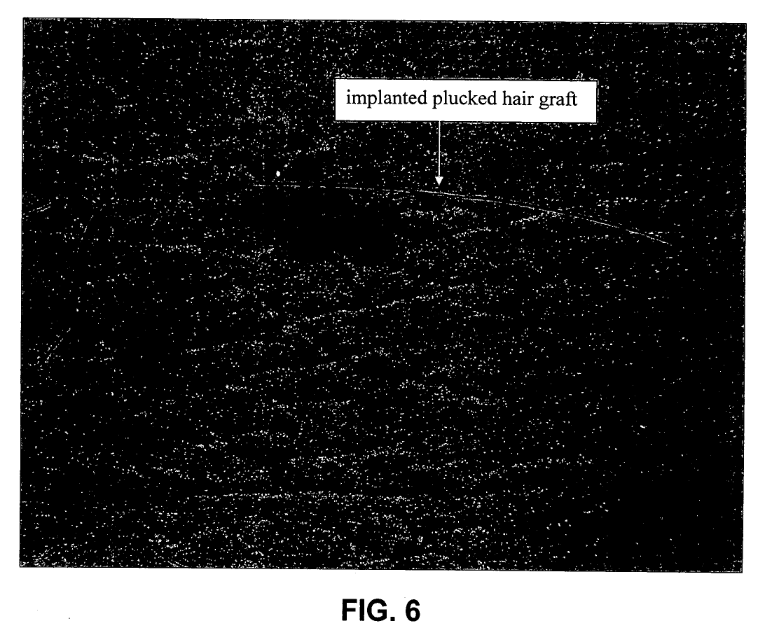

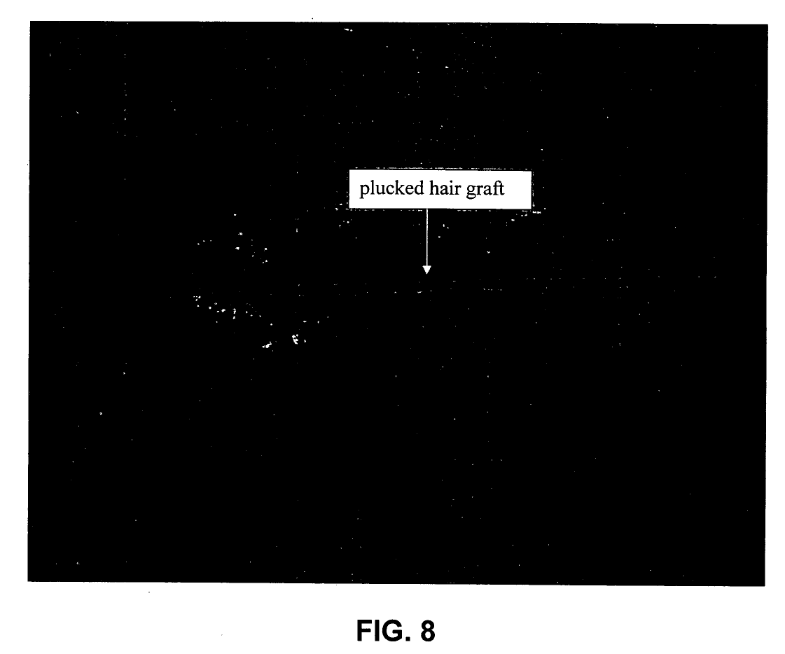

Surgery was performed on SCID and nude (nu/nu) mice as described in Example 2, except that the implanted hair grafts were allowed to reside in vivo for longer times; typically greater than 4 months. In the case of the SCID mouse, which had white fur, the hair shaft from the implanted control whole human hair graft occasionally broke through the skin and continued to elongate. See FIG. 3. Human hair grafts observed beneath the skin of both mouse types appeared equivalent. As can be seen by comparing FIG. 8 with FIG. 3, follicle neogenesis induced by the plucked hair graft (FIG. 8) was surprisingly similar in appearance to a whole follicle implant (FIG. 3).

### Example 6

Follicular Neogenesis in Swine with Implanted Cells

Purpose bred Sinclair miniature swine were used as subjects under an IACUC approved protocol in compliance with current guidelines. All procedures were conducted using aseptic surgical technique with animals under general anesthesia.

Neonatal same-breed skin was obtained and freshly processed by the method disclosed by Zheng, et al. in U.S. patent application 20060062770, “Organogenesis from dissociated cells” for isolation of dermal and epidermal cells from neonatal mouse skin. Since pig skin, like human skin, is at least 10 times thicker than mouse skin, we sought to implant cells at various levels within the dermis including the fat-dermis interface. This was accomplished by creating precisely dimensioned cavities with an erbium-YAG laser. Cells were combined with MATRIGEL™ (BD Biosciences, San Jose, Calif. USA 95131) and implanted at various depths. As a control, traditional surgical hair transplantation was performed.

Routine histological evaluation of biopsy specimens was conducted. In one case, a female adult pig was implanted with male neonatal cells and the resultant implant site evaluated for the presence of hair follicle structures containing male cells via fluorescence in situ hybridization (“FISH”) analysis using a y-chromosome specific probe. New hair follicles were detected as early as 30 days post-implantation and formed most successfully when the cells were implanted at the level of the fat-dermis interface.

In replicate studies, hair follicles formed in 8 out of 18 and in 19 out of 48 implantations. Unlike mouse follicle neogenesis from dissociated cells, which yielded dozens of individual but randomly oriented follicles per injection, the pig cells only formed a single follicle at each implant site. The detection of y-chromosome positive cells in the case where male cells were implanted in the female pig confirmed that the formed follicle contained the implanted cells. These follicles invariably produced ingrown hair shafts. However, this may be endemic to this host system since a surprising rate of ingrown hair was also observed with whole follicle transplantation.

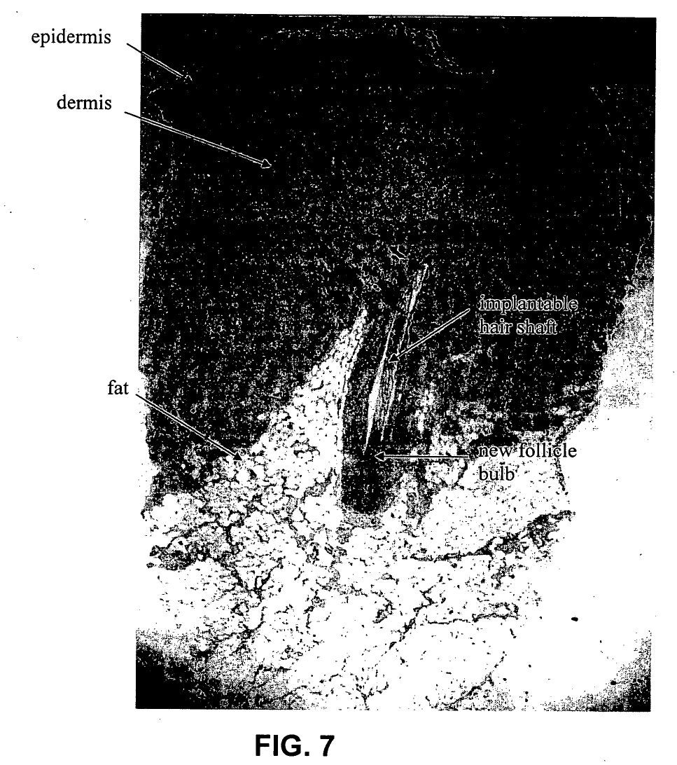

To solve the problem of ingrown hair, a plucked pig hair shaft devoid of living cells was implanted along with the trichogenic cells. After 28 days post-implantation a new bulb was observed to have formed and was oriented in the direction of the implanted hair shaft toward the epidermis, as shown in FIG. 7. Similar histological sections of all follicles formed from implanted cells without added plucked hair produced bulbs that were oriented parallel to the epidermis rather than toward it.

What is claimed is:

1. A hair graft comprising at least one plucked hair having adhered epidermal stem cells and follicular dermal cells associated with the epidermal stem cells.

2. The graft of claim 1, wherein the follicular dermal cells are dermal papilla or dermal sheath cells.

3. The graft of claim 1, further comprising a bioabsorbable scaffold.

4. The graft of claim 2, wherein the bioabsorbable scaffold is a hollow filament having an inner lumen.

5. The graft of claim 3, wherein the plucked hair is located within the inner lumen.

6. The graft of claim 3, wherein the hollow filament has one closed end.

7. The graft of claim 3, wherein the hollow filament has two open ends.

8. The graft of claim 3, further comprising a protectant contained within the inner lumen of the filament.

9. The graft of claim 7, wherein the protectant is a fibrous material, a gel-forming material or a porous material.

10. The graft of claim 7, wherein the protectant is selected from the group consisting of collagen, gelatin, cellulose derivatives, starch, dextrin, chitosan, lipoproteins, recombinant human forms of collagen and gelatin, fibrinogen, fibrin, fibronectin, laminin, albumin, serum, polysaccharides, mucopolysaccarides, and combinations thereof.

11. The graft of claim 9, wherein the gel-forming material is selected from the group consisting of collagen, gelatin, albumin, laminin, heparin sulfate proteoglycans, entactin, ethylene oxide, propylene oxide, polyethylene glycol molecules with end groups that covalently react to form gel networks and combinations thereof.

12. The graft of claim 10, wherein the gel-forming material comprises a copolymer of ethylene and propylene oxide, wherein the copolymer hydrophilically coats the inner surface to form a gel when the graft is placed in a warm environment.

13. The graft of claim 10, wherein the protectant is crosslinked.

14. The graft of claim 2, wherein the bioabsorbable scaffold further comprises at least one moiety associated with the scaffold.

15. The graft of claim 13, wherein the moiety is selected from the group consisting of a growth factor, an angiogenesis factor, a cell attachment binding site moiety, a cell signaling molecule, a small molecule, a glycoprotein, a bioactive molecule and combinations thereof.

16. A hair graft comprising at least one plucked hair having adhered epidermal stem cells, the at least one plucked hair located within a bioabsorbable scaffold, wherein the bioabsorbable scaffold contains a protectant and a plurality of follicular dermal cells.

17. A method of making a hair graft comprising at least one plucked hair from at least one hair follicle and incubating the at least one plucked hair with follicular dermal cells to form a hair graft, wherein the at least one plucked hair has adhered epidermal stem cells.

18. The method of claim 16, wherein the follicular dermal cells are dermal papilla cells.

19. The method of claim 16, wherein the at least one plucked hair and follicular dermal cells are incubated in a scaffold having an inner lumen.

20. The method of claim 19, wherein the scaffold is bioabsorbable.

21. The method of claim 19, further comprising a protectant located within the scaffold.

22. The method of claim 21, wherein the follicular dermal cells are located within the protectant.

23. A method of implanting a hair graft comprising creating a wound in skin of a subject and implanting the hair graft of claim 1 into the wound.

24. The method of claim 23, further comprising placing a protectant into the wound.

25. The method of claim 24, wherein the protectant is serum, plasma or a combination thereof.

26. The method of claim 23, wherein the hair graft further comprises a bioabsorbable scaffold.

27. The method of claim 23, wherein the bioabsorbable scaffold further comprises a moiety associated with the scaffold.

28. The method of claim 27, wherein the moiety is a selected from the group consisting of a growth factor, an angiogenesis factor, a cell attachment binding site moiety, a cell signaling molecule, a small molecule, a polypeptide, a glycoprotein, a bioactive molecule and combinations thereof.

29. The method of claim 23, wherein the at least one plucked hair is from the subject.

30. The method of claim 26, wherein the follicular dermal cells are from the subject.

31. A method of implanting a hair graft comprising
a) loading a syringe with a protectant;
b) loading the hair graft of claim 1 into a hypodermic needle; and
c) injecting the hair graft and the protectant into the subject.

32. The method of claim 31, wherein the hair graft and the protectant is injected into the uppermost fat layer of the skin.

33. A method for identifying inductive follicular dermal cells comprising incubating at least one plucked hair having adhered epidermal stem cells in follicular dermal cells, wherein the epidermal stem cells selectively attract follicular dermal cells capable of inducing follicular neogenesis.
